(19) United States
(12) Patent Application Publication
(10) Pub. No.: US 2005/0202056 A1
(43) Pub. Date: Sep. 15, 2005

(54) Composition for reducing enzymatic irritation to skin

(76) Inventors: Laura McCulloch, Hanpshire (GB); John Hopkins, Berkshire (GB)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/960,862

(22) Filed: Oct. 7, 2004

Related U.S. Application Data
(63) Continuation-in-part of application No. 10/035,454, filed on Nov. 1, 2001.

(51) Int. Cl. A61K 7/00

(52) U.S. Cl. 424/401

(57) ABSTRACT

A composition that is useful for reducing irritation to skin caused by enzymes is disclosed. The composition contains a swellable clay having layers in a lattice structure and a peptizing agent. The composition may be a solid, colloidal suspension, or emulsion formulation. Methods of treating skin to reduce or prevent irritation caused by enzymes are also disclosed.

### BACKGROUND OF THE INVENTION

1. Field of the Invention

The present invention relates to a topical composition for use in preventing, treating, or reducing skin rash, such as diaper rash, resulting from enzymatic irritation to the skin, and methods therefor.

2. Description of the Prior Art

One common skin rash is perineal dermatitis, which includes diaper dermatitis or “diaper rash.” Perineal dermatitis has been defined as contact dermatitis in the perineal area, including the perineum buttocks, and the perineal, coccyx, and upper/inner thigh regions. See Brown, D. S, et al., 39(7) A Conceptual Framework, Ostomy/Wound Management 20-25 (1993)(“Brown”). The physical signs of diaper dermatitis may include one or a combination of erythema, oozing, selling, crusting, scaling, and visiculation, with the possibility of hyperpigmentation, thickening, and excoriation over time. See Brown.

Diaper dermatitis is believed to be caused by the prolonged contact of the skin with fecal matter and urine. Although the exact component or components of urine and feces responsible for diaper dermatitis has not been identified, some possible factors include ammonia, urine pH, fecal microorganisms, and lipase and protease enzymes found in fecal matter.

Currently, the main focus of dermatitis treatments has been to reduce the exposure of the skin to such body wastes via barrier products, e.g. diaper creams and lotions. However, such barrier products tend to only address the rash symptoms. It would be preferable to have a composition that not only would address diaper rash symptoms, but would also prevent the formation of the rash at the onset.

Another mode of treatment focuses on the incorporation of agents to inhibit various fecal enzymes, such as lipase, that often aggravate perineal dermatitis. For example, WO99/26619 discloses the use of particular ester compounds capable of functioning as lipase enzyme substrates in treating diaper rash. U.S. Pat. No. 6,207,596 B1 discloses a wipe containing a particular aromatic diamidine for inhibiting protease enzymes. Alternatively, WO 97/38735 provides for the use of clays capable of deactivating such fecal enzymes. Unfortunately, some components found in urine appear to react with, and thus partially inactivate, the enzyme-inhibiting ability of such clays.

Therefore, there is a need for a composition that is effective at reducing, preventing, and/or treating perineal dermatitis such as diaper rash. Such compositions should not only be capable of reducing the irritation caused by fecal enzymes, but also remain active in the presence of urine.

### SUMMARY OF THE INVENTION

In accordance with this invention, there is provided a topical solid composition comprised of, consisting of, and/or consisting essentially of:
a. a swellable clay, and
b. a peptizing agent.

In a second aspect, the present invention provides a cosmetic composition in the form of an oil in water emulsion comprised of, consisting of, and/or consisting essentially of:
a. a swellable clay,
b. an emulsifying agent,
c. an oil, and
d. an aqueous-based component.

In a third aspect, the present invention provides a method of treating or reducing enzymatic dermatitis comprising applying to the skin of a mammal an effective amount of a topical composition comprised of, consisting of, and/or consisting essentially of:
a. a swellable clay, and
b. a peptizing agent.

In a fifth aspect, the present invention provides a method of preventing enzymatic dermatitis comprising applying to the skin of a mammal an effective amount of a topical composition comprised of, consisting of, and/or consisting essentially of:
a. a swellable clay, and
b. a peptizing agent.

In a sixth aspect, the present invention provides a method of treating or reducing skin irritation caused by enzymes comprising applying to the skin a topical composition in the form of an oil in water emulsion comprised of, consisting of, and/or consisting essentially of:
a. a swellable clay,
b. an emulsifying agent,
c. an oil, and
d. an aqueous-based component.

In a seventh aspect, the present invention provides a method of preventing skin irritation caused by enzymes comprising applying to the skin a topical composition in the form of an oil in water emulsion comprised of, consisting of, and/or consisting essentially of:
a. a swellable clay,
b. an emulsifying agent,
c. an oil, and
d. an aqueous-based component.

In a final aspect, the present invention provides a cosmetic composition in the form of a water in oil emulsion comprised of, consisting of, and/or consisting essentially of:
a. a swellable clay,
b. an emulsifying agent,
c. an oil,
d. an aqueous-based component, and
e. a peptizing agent,
   as well as the use of such a cosmetic composition to prevent, treat, and/or reduce enzymatic dermatitis or skin irritation.

The invention also provides for a disposable absorbent article containing the compositions as described above.

We have unexpectedly discovered a composition that effectively reduces and/or treats the skin irritation caused by fecal enzymes, prevents the development of perineal dermatitis, and also which is not inactivated in the presence of urine.

### DETAILED DESCRIPTION OF PREFERRED EMBODIMENTS

The compositions of the present invention are useful in the prevention, reduction or treatment of enzymatic dermatitis, such as perineal dermatitis, of the external skin.

By “treatment” or “reduction” is meant herein the reduction of the dermatitis or the rash of the skin which is caused by the presence of fecal enzymes on the skin, or at least the stabilizing of the dermatitis or rash of the skin that is caused by these enzymes.

By “affected area” is meant the area of skin that is presently exhibiting any levels of skin rash or enzymatic dermatitis, or the area that will be in prolonged contact with feces containing such dermatitis-causing enzymes. This area also includes the area immediately proximate to the described area. It is the area at which treatment, reduction of, and/or prevention is desired.

The first component of the composition of the present invention is a swellable clay or adduct thereof. By the term “swellable clay,” it is meant a clay having weakly bound ions in interlayer positions that may be hydrated or may absorb organic solvents. These clays generally possess a low cationic or anionic charge, i.e. less than about 0.9 units of charge per unit cell.

By the term “adducts,” it is meant the oil swellable clays, i.e. those that swell in organic, non-aqueous solvents such as polar and nonpolar solvents, that may be prepared by reacting a water swellable clay with an organic material that binds to the clay. Examples of such binding organic materials include, but are not limited to, a quaternary ammonium compound having the structure:
R₁R₂R₃R₄N+X—
wherein
R₁, R₂, R₃ and R₄ are each independently selected from H, a C₁ to C₂₂ alkyl, a C₁ to C₂₂ alkenyl, and a C₁ to C₂₂ aralkyl, provided that at least one of the R groups is such an alkyl, alkenyl or aralkyl; and
X is the water swellable clay.

The swellable clays useful in the present invention may be structured, or may be a mixture of both structured components and amphorous components. As used herein, the terms “amorphous clay” shall include any clay without an ordered structure. Examples of such amorphous clays include, but are not limited to, allophane, imogolite, and mixtures thereof.

Examples of various structures that may be present in the clay include sheets or layers, wherein a combination of such layers is referred to as a lattice structure. Examples of suitable clay lattice structures include the pyrophillite (dioctahedral) type, the talc (trioctahedral) type, or mixtures thereof. Classes of suitable structured swellable clays include, but are not limited to the smectite clays, sepiolite clays, zeolite clays, palygorskite clays, or mixtures thereof. Examples of suitable smectite clays that are useful in this invention include, but are not limited to, the aluminosilicate clays such as bentonite, montmorillonite, hectorite, sucinite, saponite, nontronite, vermiculite, beidellite, stevensite, and their synthetically made counterparts and mixtures thereof. Montmorillonite clay is preferred. See U.S. Pat. No. 5,869,033, which is incorporated by reference herein.

In one embodiment, the clays may possess a multi-layer structure, wherein at least one layer is comprised of a smectite clay or a mixture thereof. Other clays that may be either mixed with the smectite clay in a given layer or may comprise the other layers include, but are not limited to, sepiolite, palygorskite, zeolites, and mixtures thereof.

The composition of the present invention may be in the form of a solid, a colloidal suspension, a water in oil emulsion, or an oil in water emulsion.

The amount of clay useful in this invention will depend on the desired form of the final composition. For example, in solid compositions, the amount of clay may range, based upon the total weight of the composition, from about 0.1% to about 99.9%, for example from about 2% to about 50%, and from about 2% to about 10%. For colloidal suspension compositions, the amount of clay may range, based upon the total weight of the composition, from about 0.1% to about 20%, for example from about 0.5% to about 10% and from about 1% to about 5%. For emulsion compositions, the amount of clay may range, based upon the total weight of the composition, from about 0.1% to about 10%, for example from about 1% to about 5% and from about 1% to about 3%.

The second component of the solid or suspension composition of the present invention is a peptizing agent, which is used in an amount effective for overcoming the problem of clay inactivation upon exposure to urine. Suitable peptizing agents may be selected from tetrasodium pyrophosphate, tetrapotassium pyrophosphate, sodium hexametaphosphate, sodium tripolyphosphate, ethylenediamine tetracetic acid and its derivatives, sodium silicate, sodium oxalate, sodium hydroxide, sodium carbonate, sodium polyacrylate, hydrogen peroxide, sodium citrate, alkylamido betaines, alkyl betaines, and mixtures thereof, wherein the alkyl contains from about 8 carbon atoms to about 22 carbon atoms. Tetrasodium pyrophosphate and tetrapotassium pyrophosphate are preferred.

In general, the solid composition of the present invention contains the peptizing agent in an amount, based upon the total weight of the solid composition, from about 0.05% to about 10%, for example from about 0.1% to about 5% and from about 0.1% to about 1%. For compositions in the form of a colloidal suspension, the peptizing agent may be added at an amount sufficient to enable the clay to form a stable colloidal suspension. The weight ratio of swellable clay to peptizing agent may range from about 10:1 to about 1:10, for example from about 5:1 to about 1:5, and from about 5:1 to about 1:1. For compositions in the form of an emulsion, the peptizing agent may optionally be added thereto such that the weight ratio of swellable clay to peptizing agent may range from about 10:1 to about 1:10, for example from about 5:1 to about 1:5, and from about 5:1 to about 1:1. For solid compositions of the present invention, the balance of the composition may optionally be comprised of other materials such as fillers, active ingredients, absorbent polymers, fragrances, and the like in amounts typically used in the art. See U.S. Pat. No. 5,969,033, which is incorporated by reference herein.

For colloidal suspension compositions of the present invention, the composition is generally further comprised of an aqueous component such as water in an amount, based upon the total weight of the composition, of from about 50% to about 99.9%, for example from about 60% to about 99.9% and from about 70% to about 99.9%. These colloidal suspension compositions may also be comprised of fillers, active ingredients, fragrances, and the like in the amounts typically used in the art.

For emulsion compositions of the present invention, the composition is comprised of, based upon the total weight of the composition, from about 1 percent to about 50 percent, for example, from about 5 percent to about 20 percent of at least one oil, from about 50 percent to about 99 percent, for example, from about 80 percent to about 95 percent of at least one aqueous component, from about 0.1 percent to about 20 percent, for example, from about 1 percent to about 5 percent of a swellable clay, and for oil in water emulsions, from about 0 percent to about 10 percent, for example, from about 0.05 percent to about 3 percent of a peptizing agent, but for water in oil emulsions from about 0.05 percent to about 3 percent of a peptizing agent. The amount of emulsifying agent used may depend upon several factors such as the type of system to be emulsified and the type of emulsifier selected; however, in general, the composition may contain, based upon the total weight of the composition, from about 0.5 percent to about 10 percent, for example, from about 1 percent to about 5 percent of at least one emulsifying agent. The emulsion may be in the form of a lotion, a milk, or a cream.

The oil in water emulsion composition contains, based upon the total weight of the composition, from about 0.1% to about 50%, for example from about 0.1% to about 20% and from about 0.1% to about 10% of an oil phase, and from about 50% to about 99.9%, for example from about 80% to about 99.9% and from about 90% to about 99.9% of a water phase.

The oil phase may be comprised of any oil. Examples of suitable oils include, but are not limited to, mineral oil, lanolin, vegetable oils, and mixtures thereof.

The water phase may be comprised of an aqueous component including water, glycols such as propylene glycol, glycerin, dipropylene glycol, and mixtures thereof.

Emulsifying agents that are predominantly hydrophilic in nature are typically added to the aqueous phase, while emulsifying agents that are predominantly lipophilic in nature are typically added to the oil phase. Emulsifying agents suitable for use in the present invention may be at least one nonionic, anionic, cationic, or amphoteric surfactant. Combinations of more than one nonionic, anionic, cationic, or amphoteric surfactant may also be useful. The type of surfactants utilized and the hydrophobe/lypophobe balance of the surfactants utilized will depend on the type of oil-in-water composition desired. It is within the expertise of those of ordinary skill in the art to select surfactant combinations that will provide the desired properties. The amount of emulsifying agent in the oil-in water emulsion composition of the present invention is dependent on the particular class of emulsifier and the respective levels of the oil and the aqueous phases, but in general is present in an amount, based upon the total weight of the composition, from about 0.1% to about 20%.

Nonionic surfactants useful in this invention include ethoxylated fatty alcohols, ethylene oxide/propylene oxide block copolymers, ethoxylated alkylphenols, ethoxylated or non-ethoxylated esters of alkyl glucose and fatty acid, and glycerol esters.

One class of nonionic surfactants useful in the present invention are polyoxyethylene derivatives of polyol esters, wherein the polyoxyethylene derivative of polyol ester (1) is derived from (a) a fatty acid containing from about 8 to about 22, and preferably from about 10 to about 14 carbon atoms, and (b) a polyol selected from sorbitol, sorbitan, glucose, α-methyl glucoside, polyglucose having an average of about 1 to about 3 glucose residues per molecule, glycerine, pentaerythritol and mixtures thereof, (2) contains an average of from about 10 to about 120, and preferably about 20 to about 80 oxyethylene units; and (3) has an average of about 1 to about 3 fatty acid residues per mole of polyoxyethylene derivative of polyol ester.

Examples of polyoxyethylene derivatives of polyol esters include, but are not limited to PEG-80 sorbitan laurate and Polysorbate 20. PEG-80 sorbitan laurate, which is a sorbitan monoester of lauric acid ethoxylated with an average of about 80 moles of ethylene oxide, is available commercially from Uniqema Americas of Wilmington, Del. under the tradename, “Atlas G-4280.” Polysorbate 20, which is the laurate monoester of a mixture of sorbitol and sorbitol anhydrides condensed with approximately 20 moles of ethylene oxide, is available commercially from Uniqema Americas of Wilmington, Del. under the trade name “Tween 20.”

Another class of suitable nonionic surfactants includes long chain alkyl glucosides or polyglucosides, which are the condensation products of (a) a long chain alcohol containing from about 6 to about 22, and preferably from about 8 to about 14 carbon atoms, with (b) glucose or a glucose-containing polymer. The alkyl glucosides have about 1 to about 6 glucose residues per molecule of alkyl glucoside.

As used herein, the term “amphoteric” shall mean: 1) molecules that contain both acidic and basic sites such as, for example, an amino acid containing both amino (basic) and acid (e.g., carboxylic acid, acidic) functional groups; or 2) zwitterionic molecules which possess both positive and negative charges within the same molecule. The charges of the latter may be either dependent on or independent of the pH of the composition. Examples of zwitterionic materials include, but are not limited to, alkyl betaines and amidoalkyl betaines. The amphoteric surfactants are disclosed herein without a counter ion. One skilled in the art would readily recognize that under the pH conditions of the compositions of the present invention, the amphoteric surfactants are either electrically neutral by virtue of having balancing positive and negative charges, or they have counter ions such as alkali metal, alkaline earth, or ammonium counter ions.

In embodiments wherein the emulsifying agents are alkyl betaine, amidoalkyl betaine, or mixtures thereof, the emulsion may further contain additional amounts of alkyl betaine, amidoalkyl betaine, or mixtures thereof, in amounts in excess of that which is needed to emulsify the suspension, for peptizing agent purposes.

Commercially available amphoteric surfactants are suitable for use in the present invention and include, but are not limited to amphocarboxylates, alkyl betaines, amidoalkyl betaines, amidoalkyl sultaines, amphophosphates, phosphobetaines, pyrophosphobetaines, carboxyalkyl alkyl polyamines, and mixtures thereof.

Anionic surfactants are useful in the present invention and may be selected from the following classes of surfactants: alkyl sulfates; alkyl ether sulfates; alkyl monoglyceryl ether sulfates; alkyl monoglyceride sulfates; alkyl monoglyceride sulfonates; alkyl sulfonates; alkylaryl sulfonates; alkyl sulfosuccinates; alkyl ether sulfosuccinates; alkyl sulfosuccinamates; alkyl amidosulfosuccinates; alkyl carboxylates; alkyl amidoethercarboxylates; alkyl succinates; fatty acyl sarcosinates; fatty acyl amino acids; fatty acyl taurates; fatty alkyl sulfoacetates; fatty acids; alkyl phosphates; and mixtures thereof.

The compositions of the present invention may also include one or more optional ingredients nonexclusively including a pearlescent or opacifying agent, a thickening agent, secondary conditioners, humectants, chelating agents, and additives which enhance their appearance, feel and fragrance, such as colorants, fragrances, preservatives, pH adjusting agents, skin conditioners, anti-irritants, anti-inflammatories, anti-microbial agents, sensates, anti-puritics, skin protectants, film formers, preservatives, and the like. The pH of the compositions of this invention is preferably maintained in the range of about 2 to about 10, preferably from about 4 to about 9, and most preferably from about 7 to about 9.

Commercially available pearlescent or opacifying agents which are capable of suspending water insoluble additives are suitable for use in this invention. The pearlescent or opacifying agent is present in an amount, based upon the total weight of the composition, of from about 0 percent to about 3 percent, preferably from about 0.25 percent to about 2.5 percent, and more preferably, from about 0.5 percent to about 1.5 percent. Examples of suitable pearlescent or opacifying agents include, but are not limited to
a) mono or diesters of (1) fatty acids having from about 16 to about 22 carbon atoms and (2) either ethylene or propylene glycol;
b) mono or diesters of (1) fatty acids having from about 16 to about 22 carbon atoms (2) a polyalkylene glycol of the formula:
   HO-(JO)ₐ—H,
wherein
J is an alkylene group having from about 2 to about 3 carbon atoms;
and a is 2 or 3;
c) fatty alcohols containing from about 16 to about 22 carbon atoms;
d) fatty esters of the formula: KCOOCH₂L, wherein K and L independently contain from about 15 to about 21 carbon atoms;
e) inorganic solids insoluble in the composition, and
f) mixtures thereof.

The pearlescent or opacifying agent may be introduced to the composition as a pre-formed, stabilized aqueous dispersion, such as that commercially available from Henkel Corporation of Hoboken, N.J. under the tradename, “Euperlan PK-3000.” This material is a combination of glycol distearate (the diester of ethylene glycol and stearic acid), Laureth-4 (CH₃(CH₂)₁₀CH₂(OCH₂CH₂)₄OH) and cocamidopropyl betaine and preferably is in a weight percent ratio of from about 25 to about 30: about 3 to about 15: about 20 to about 25, respectively.

Commercially available thickening agents that are capable of imparting the appropriate viscosity to the compositions may be suitable for use in this invention. If used, the thickener should be present in the compositions in an amount sufficient to raise the Brookfield viscosity of the composition to a value of between about 500 to about 10,000 centipoise. Examples of suitable thickening agents nonexclusively include:
a) mono or diesters of 1) polyethylene glycol of formula:
   HO—(CH₂CH₂O)ₙH,
wherein z is an integer from about 3 to about 200; and 2) fatty acids containing from about 16 to about 22 carbon atoms;
b) fatty acid esters of ethoxylated polyols;
c) ethoxylated derivatives of mono and diesters of fatty acids and glycerine;
d) hydroxyalkyl cellulose;
e) alkyl cellulose;
f) hydroxyalkyl alkyl cellulose; and
g) mixtures thereof. Preferred thickeners include polyethylene glycol ester, and more preferably PEG-150 distearate which is available from the Stepan Company of Northfield, Ill. or from Comiel, S.p.A. of Bologna, Italy under the trade name, “PEG 6000 DS”.

Commercially available humectants, which are capable of providing moisturization and conditioning properties to the composition, are suitable for use in the present invention. The humectant is present in an amount of from about 0 percent to about 10 percent, preferably from about 0.5 percent to about 5 percent, and more preferably from about 0.5 percent to about 3 percent, based on the overall weight of the composition. Examples of suitable humectants nonexclusively include: 1) water soluble liquid polyols selected from the group comprising glycerine, propylene glycol, hexylene glycol, butylene glycol, dipropylene glycol, and mixtures thereof; 2) polyalkylene glycol of the formula:
HO—(R″O)_{b}—H,
wherein R″ is an alkylene group having from about 2 to about 3 carbon atoms and b is an integer of from about 2 to about 10;
3) polyethylene glycol ether of methyl glucose of formula
   CH₃—C₆H₁₀O₅—(OCH₂CH₂)_{c}—OH,
wherein c is an integer from about 5 to about 25;
   4) urea; and 5) mixtures thereof, with glycerine being the preferred humectant.

Preservatives may be useful additives to the formulations of this invention. Suitable preservatives include Quaternium-15, available commercially as “Dowicil 200” from the Dow Chemical Corporation of Midland, Mich., and are present in the composition in an amount, based upon the total weight of the composition, from about 0 to about 0.2 percent, and preferably from about 0.05 percent to about 0.10 percent.

The cosmetic compositions of the present invention may be in the form of a cream, lotion, gel, foam, oil, ointment, or powder for topical application to the external skin. The compositions of the present invention may be in a form suitable for application to the skin in either a leave-on product or a rinse-off product.

The above described composition may be prepared by combining the desired components in a suitable container and mixing them under ambient conditions in any conventional mixing means well known in the art, such as a mechanically stirred propeller, paddle, and the like. Although the order of mixing is not critical, it is preferable to pre-blend certain components, such as the fragrance and the nonionic surfactant before adding such components into the main mixture.

When using a thickener component, it is also preferable to preblend the desired thickener with from about 5 percent to about 20 percent, based upon the total weight of the composition, of water and preferably at a temperature of from about 60° C. to about 80° C. When processing with a thickener, it is also preferable to reduce the temperature of the overall composition to less than about 45° C. before any pre-formed pearlizer is added thereto.

We have unexpectedly found that the swellable clay-containing compositions of the present invention are effective in reducing the enzymatic activity of the enzymes present on the external skin.

Another embodiment of the present invention is directed to methods for treating, reducing and/preventing enzymatic-based dermatitis/diaper rash/skin rash using an effective amount of the compositions described above.

Although the “effective amount” of composition used to treat, reduce, or prevent the rash will depend upon, for example, the severity of skin irritation at the affected area, the concentration of enzymes in the exudate, and the like, typically for reducing, treating, or preventing purposes, from about 0.1 mg/square cm to about 3 mg/square cm, for example from about 1 mg/square cm to about 2.5 mg/square cm of the composition, is applied to the affected area.

The methods of the present invention may be used to treat or reduce rashes caused by any one or more of the enzymes associated with diaper rash including, for example, lipases, such as lipase, proteases such as trypsin, chymotrypsin, elastase, carboxypeptidase, aminopeptidase, as well as, esterases, ureases, combinations of two or more thereof, and the like.

The compositions of the present invention may be applied directly to the affected area of the skin or via a substrate such as a wipe, diaper liner or facing to such affected area.

Although the structure of the substrate is not critical to the practice of the present invention, examples of suitable substrates are disclosed in, for example, U.S. Pat. No. 6,204,596 and WO99/26619, which are incorporated by reference herein.

The substrate may have the composition incorporated therein, or alternatively the composition may be provided separately from the substrate and applied thereto at the time of use. The amount of composition incorporated into the substrate is an amount effective for delivering the required treatment, reduction and/or prevention of the dermatitis. In one embodiment, the substrate contains the composition at such a level that the composition is present therein at a level of, based upon the total weight of the substrate, from about 35 g/square meter to about 90 g/square meter for example from about 50 g/square meter to 75 g/square meter.

The invention illustratively disclosed herein suitably may be practiced in the absence of any component, ingredient, or step which is not specifically disclosed herein. Several examples are set forth below to further illustrate the nature of the invention and the manner of carrying it out. However, the invention should not be considered as being limited to the details thereof.

### EXAMPLES

### Example 1

### Preparation of Clay-Containing Dispersion

A. Preparation of Water Phase:

320.4 g of sterile water was added to a 600 ml beaker. After adding 1.2 g of tetrasodium pyrophosphate (“TSPP”) thereto with mixing 10 minutes, 12 g of sodium magnesium silicate available from Southern Clay Products under the tradename, “Laponite® XLS” (“XLS”) was added thereto with stirring for 15 minutes under ambient conditions.

240 g of deionized water were added to a second 800 ml beaker. 1.2 g of Acrylates/C10-C30 Alkyl Acrylate Crosspolymer available from BF Goodrich under the tradename, “Pemulen® TR1” (“Pemulen”) was added thereto with stirring with a 6 hole paddle for 20 minutes, then the mixture was cooled to room temperature. The pH of the solution was adjusted to 7.0 with sodium hydroxide.

After both mixtures were combined and mixed for 15 minutes, 18 g of mineral oil were added thereto with mixing for 20 minutes. 3 g of ethylenediamine tetracetic acid (‘EDTA’) available from the Dow Chemical Company under the tradename, “Versene® 100XL” were added thereto, then the mixture was homogenized with a Gifford-Wood homogenizer for 15 minutes.

### Example 2

### Preparation of Clay-Containing Water in Oil Emulsion

After 605.7 g of sterile water were heated to a temperature of about 40° C. on a hot plate, 1.8 g of TSPP were added thereto over 4 minutes. 4.5 g of EDTA were then added thereto. After heating the solution to 65° C., 18 g of the sodium magnesium silicate of Example 1 was added there to with mixing for 15 to 30 minutes to yield a water phase mixture.

225 g of mineral oil available from Penreco Company under the tradename, “Drakeol® 7” were combined with 45 g of PEG-30 dipolyhydroxy stearate available from the Uniqema Americas Company under the tradename, “Arlacel® P135” in a beaker on a hot plate. The solution was then mixed for 10 minutes at a temperature of 65° C. to yield an oil phase.

The water phase was then added to the oil phase and mixed for 15 minutes at 65° C., then homogenized with a Gifford-Wood homogenizer for 20 minutes at 40% maximum speed to form an emulsion. The resulting emulsion was exposed to a vacuum pressure atmosphere for about 4 minutes, then the emulsion was cooled to room temperature.

### Example 3

### Preparation of Clay-Containing Oil in Water Emulsion

2 g of TSPP were added into a beaker containing 443 g deionized water with mixing for 10 minutes via a mixer available from the Caframo Company under the tradename, “Lightnin®.” 25 g of the sodium magnesium silicate of Example 1 were slowly added thereto with constant mixing until the mixture was clear and homogenous. The mixture was then heated to 73° C. to yield Phase A.

15 g of the mineral oil of Example 2. Were added to a second beaker and heated to 75° C. 9.75 g of steareth-21 available through Uniqema Americas Company under the tradename, “Brij® 721.” and 5.25 g of steareth-2 available through Uniqema Americas Company under the tradename, Brij® 72 (were then added thereto with mixing to yield Phase B.

Phase B was slowly added to Phase A at 75° C. with mixing until a homogeneous emulsion was formed.

### Example 4

### Preparation of a Clay-Containing Surfactant Composition

10 g of the silicate of Example 3 were slowly added into a beaker containing 453.2 g deionized water with mixing until the mixture was somewhat clear and the silicate was completely dispersed. After 33.3 g of cocamidopropyl betaine (30%) available from the Goldschmidt Company under the tradename, “Tegobetaine L-7” was added thereto with mixing for 5 minutes, then 1 g of TSPP was added thereto. 2.5 g of the EDTA of Example 3 were then added thereto.

### Example 5

### Enzymatic Efficacy Comparisons

The clay formulations as set forth in Table A below were prepared generally in accordance with the procedures set forth in Examples 1 to 4 above.

The values of the formulation ingredients are expressed in terms of weight percent, with q.s. water.

TABLE A
Clay-Containing Formulations

| | | | | | Form: |
|---|---|---|---|---|---|
| | | | (wt %) | | (Suspension/ |
| Sample | (wt %) | | Peptizing | | Emulsion/ |
| No. | Clay | (wt %) Emulsifier* | Agent | Other | Solid) |
| 1 | 2 LAPONITE | 0.2 Pemulen TR1 | 0.2 TSPP; | 3% | Oil-in water |
| | XLS | | 0.5 EDTA | mineral oil | emulsion |
| 2 | 5 LAPONITE | 1.05 steareth 2¹; | 0.4 TSPP | 3% | Oil-in water |
| | XLS | 1.95 steareth 21² | | mineral oil | emulsion |
| 3 | 2 LAPONITE | 2 Arlacel P135³ | 0.2 TSPP; | 3% | Oil-in water |
| | XLS | | 0.5 EDTA | mineral oil | emulsion |
| 4 | 2 LAPONITE | 2 Emulgade SE-PF⁴; | — | 3% | Oil-in water |
| | XLS | 0.94 Emulgin B1⁵ | | mineral oil | emulsion |
| 5 | 2 Q18⁶ | 0.2 Pemulen TR1⁷ | 0.2 TSPP; | 3% | Oil-in water |
| | Bentonite | | 0.5 EDTA | mineral oil | emulsion |
| 6 | 2 Q18 | 0.2 Pemulen TR1 | — | 3% | Oil-in water |
| | Bentonite | | | mineral oil | emulsion |
| 7 | LAPONITE | — | — | PH of | Suspension/gel |
| | XLG⁸ | | | formulation | |
| | | | | is 7.7 | |
| 8 | 2 LAPONITE | — | — | synthetic | Suspension/gel |
| | XLG | | | urine added; | |
| | | | | pH of | |
| | | | | formulation is | |
| | | | | 7.7 | |
| 9 | 2 LAPONITE | — | 0.2 TSPP; | synthetic | Suspension/gel |
| | XLG | | 0.5 EDTA | urine added; | |
| | | | | pH of | |
| | | | | formulation is | |
| | | | | 7.7 | |
| 10 | 0.1 | — | 0.2 TSPP; | | Suspension/gel |
| | LAPONITE | | 0.5 EDTA | | |
| | XLG | | | | |
| 11 | 99.9 | — | 0.2 TSPP; | | Solid |
| | LAPONITE | | 0.5 EDTA | | |
| | XLG | | | | |
| 12 | 2 LAPONITE | — | 0.2 TSPP; | | Suspension/gel |
| | XLG | | 0.5 EDTA | | |
| 13 | 50 | — | 0.2 TSPP; | | Suspension/gel |
| | LAPONITE | | 0.5 EDTA | | |
| | XLG | | | | |
| 14 | 10 | — | 0.2 TSPP; | | Suspension/gel |
| | LAPONITE | | 0.5 EDTA | | |
| | XLG | | | | |
| 15 | 2 Q18 | 0.2 Pemulen TR1 | — | 3% mineral | Oil-in-water |
| | Bentonite | | | oil | emulsion |
| 16 | 2 LAPONITE | — | 0.2 TSPP | | Suspension/gel |
| | XLG | | | | |
| 17 | 2 LAPONITE | — | 0.2 TSPP | | Suspension/gel |
| | XLG | | | | |
| 18 | 2 LAPONITE | — | 0.2 TSPP | | Suspension/gel |
| | XLG | | | | |
| 19 | 2 LAPONITE | — | 10 TSPP | | Suspension/ |
| | XLG | | | | gel |
| 20 | 2 LAPONITE | 2 Sodium laureth | 0.2 TSPP; | | suspension/ |
| | XLG | sulfate | 0.5 EDTA | | gel |
| 21 | 2 LAPONITE | 2 stearic acid | 0.2 TSPP; | 3% mineral | Oil-in-water |
| | XLG | | 0.5 EDTA | oil | emulsion |
| 22 | 2 LAPONITE | 2% active CAPB⁹ | 0.2 TSPP; | | Suspension/ |
| | XLG | | 0.5 EDTA | | gel |
| 23 | 2 LAPONITE | — | 0.2 TSPP; | 2 amphoteric | Suspension/ |
| | XLG | | 0.5 EDTA | guar | gel |
| | | | | thickener¹⁰ | |
| 24 | 2 LAPONITE | 2 stearamidopropyl | 0.2 TSPP; | 3% mineral | Oil-in-water |
| | XLG | PG dimonium | 0.5 EDTA | oil | emulsion |
| | | chloride phosphate | | | |
| | | and cetyl alcohol¹¹ | | | |
| 25 | 2 LAPONITE | — | 0.2 TSPP; | 2% cationic | Suspension/ |
| | XLG | | 0.5 EDTA | guar | gel |
| | | | | thickener¹² | |
| 26 | 2 LAPONITE | 2 decyl polyglucose¹³ | 0.2 TSPP; | | Suspension/ |
| | XLG | | 0.5 EDTA | | gel |
| 27 | 2 LAPONITE | 2 Span 80/Tween 80 | 0.2 TSPP; | 15% mineral | Water-in-oil |
| | XLG | (70:30)¹⁴ | 0.5 EDTA | oil | emulsion |
| 28 | 2 LAPONITE | 2 Arlacel 20¹⁵ | 0.2 TSPP; | 15% mineral | Water-in-oil |
| | XLG | | 0.5 EDTA | oil | emulsion |
| 29 | 2 LAPONITE | 2 Pluronic | 0.2 TSPP; | | Suspension/ |
| | XLG | L35/Pluronic L 31¹⁶ | 0.5 EDTA | | gel |
| | | (40:60) | | | |
| 30 | 2 LAPONITE | 2 Triton 100/Triton 35 | 0.2 TSPP; | | Suspension/ |
| | XLG | (55:45)¹⁷ | 0.5 EDTA | | gel |
| 31 | 2 LAPONITE | 2 Glucam E20 | 0.2 TSPP; | 3% mineral | Oil-in-water |
| | XLG | distearate/Glucate | 0.5 EDTA | oil | emulsion |
| | | SS (75:25)¹⁸ | | | |
| 32 | 2 LAPONITE | 2 Arlacel 165¹⁹ | 0.2 TSPP; | 15% mineral | Water-in-oil |
| | XLG | | 0.5 EDTA | oil | emulsion |
| 33 | 2 LAPONITE | 0.2 Pemulen TR1 | 0.2 TSPP; | 3% mineral | Oil-in-water |
| | XLG | | 0.5 EDTA | oil, | emulsion |
| | | | | 1% capric/ | |
| | | | | caprylic | |
| | | | | triglyceride**²⁰ | |
| 34 | 2 LAPONITE | 0.2 Pemulen TR1 | 0.2 TSPP; | 3% mineral | Oil-in-water |
| | XLG | | 0.5 EDTA | oil, 1% | emulsion |
| | | | | isopropyl | |
| | | | | myristate**²¹ | |
| 35 | 2 LAPONITE | 0.2 Pemulen TR1; | 0.2 TSPP; | 3% mineral | Oil-in-water |
| | XLG | | 0.5 EDTA | oil, 1% | emulsion |
| | | | | PPG-15 | |
| | | | | stearyl ether²² | |
| 36 | 2 LAPONITE | 0.2 Pemulen TR1 | 0.2 TSPP; | 1% C11-13 | Oil-in-water |
| | XLG | | 0.5 EDTA | isoparaffin²³ | emulsion |
| | | | | (oil phase) | |
| 37 | 5.25 | 2 sorbitan stearate; | | Oil phase: | Water in oil |
| | Bentonite²⁷ | 0.30 steareth 20 | | 40% | emulsion |
| | Q18 | | | petrolatum; | |
| | | | | and 2% | |
| | | | | lauryl | |
| | | | | methicone | |
| | | | | copolyol²⁴ | |
| 38 | 2 LAPONITE | 2 sorbitan stearate; | | 3% mineral | Oil-in-water |
| | XLG | 0.3 steareth 20²⁸ | | oil and 2% | emulsion |
| | | | | lauryl | |
| | | | | methicone | |
| | | | | copolyol | |
| 39 | 2 LAPONITE | 1.95 Brij 721²⁵; | 0.2 TSPP | 3% mineral | Oil-in-water |
| | XLG | 1.05 Brij 72²⁶ | | oil | emulsion |
| 40 | 2 LAPONITE | 3 sorbitan oleate and | 0.2 TSPP; | 3% mineral | Oil-in-water |
| | XLG | polysorbate 80 | 0.5 EDTA | oil | emulsion |
| | | (70:30) | | | |

*where the total amount of an emulsifier combination is specified, the relative weight percentages of each respective component is provided in parentheses
**cosmetic esters commonly used in aesthetically pleasing personal care products
¹steareth 2 is commercially available from Uniqema Americas under the tradename, “Brij 72”;
²steareth 21 is is commercially available from Uniqema Americasunder the tradename, “Brij 721”;
³polyethylene glycol dipolyhydroxystearate is commercially available from Uniqema Americas under the tradename, “Arlacel P135;”;
⁴_Glyceryl Stearate, Ceteareth-20, Ceteareth-12, Cetearyl Alcohol, Cetyl Palmitate is commercially available from Cognis under the tradename, “Emulgade SE-PF;”
⁵Ceteareth-12 is commercially available from Cognis under the tradename, “Emulgin B1;”
⁶Q18 Bentonite is a bentonite clay available from Southern Clay Products;
⁷Acrylates/C10-C30 Alkyl acrylate crosspolymer is commercially available from BF Goodrich Company under the tradename, “Pemulen TR1;”
⁸Laponite XLG is a synthetic montmorillonite clay commercially available from Southern Clay Products;
⁹CAPB is cocamidopropyl betaine, which is commercially availahble from Goldschmidt under the tradename, “Tego Betaine L7;”
¹⁰amphoteric guar thickener available from Rhodia, Inc.
¹¹stearamidopropyl PG dimonium chloride phosphate and cetyl alcohol is available from Uniqema Americas under the tradename, “Phospholipid SV;”
¹²Guar hydroxypropyltrimonium chloride is a cationic guar thickener available from Rhone - Poulenc under the tradename, “Jaguar C17;”
¹³Decyl polyglucose is a alkyl polyglucoside available from Cognis under the tradename, “Plantaren 2000;”
¹⁴sorbitan oleate/polysorbate 80 is commercially available from Uniqema Americas under the tradename, “Span 80/Tween 80;”
¹⁵Sorbitan Laurate is commercially available from Uniqema Americasunder the tradename, “Arlacel 20;”
¹⁶poloxamer 105 is commercially available from BASF under the tradename, “Pluronic L35,” and poloxamer 101 is commercially available from BASF under the tradename, “Pluronic L31;”
¹⁷octoxynol 9 is commercially available from Union Carbide under the tradename, “Triton 100,” and octoxynol 3 is commercially available from Union Carbide under the tradename, “Triton 35;”
¹⁸polyethylene glycol methyl glucose distearate is commercially available from Croda under the tradename, “Glucam E20” and methyl glucose sesquistearate is commercially available from Croda under the tradename, “Glucate SS;”
¹⁹Glyceryl Stearate & PEG 100 Stearate is commercially available from Uniqema Americas under the tradename, “Arlacel 165;”
²⁰is commercially available from Condea Chemie under the tradename, “Miglyol 812;”
²¹isopropyl myristate is commercially available from Croda under the tradename, “Crodamol IPM;”
²²PPG-15 stearyl ether is commercially available from Uniqema Americasunder the tradename, “Arlamol E;”
²³is commercially available from Exxon Chemical under the tradename, “Isopar L;”
²⁴is commercially available from Dow Corning_under the tradename, “Q-5200 Formulation Aid;”
²⁵is steareth 21, which is commercially available from Uniqema Americas under the tradename, “Brij 721;”
²⁶is steareth 2, which is commercially available from Uniqema Americas under the tradename, “Brij 72;”
²⁷is a clay that is commercially available from Southern Clay Products;
²⁸is Steareth 20, which is commercially available from Uniqema Americas under the tradename, “Brij 78”

The clay formulations set forth above were then tested for efficacy versus lipase, a known enzyme found in feces that is responsible for causing diaper rash.

A. Preparation of Enzymatic Assay of Lipase

The enyzmatic assay of lipase was prepared in general accordance with the Sigma EC 3.1.1.3 procedure, as set forth in “Preparation of Enzymatic Assay of Lipase,” Sima-Aldrich Chemical Catalogue, pp 605 (2000/2001), which is incorporated by reference herein.

The following ingredients were used to prepare the assay:

TABLE B
Assay Components

| Reagent number | Name of Component |
|---|---|
| A | 200 mM Tris HCI Buffer, pH 7.7 @ 37° C. |
| B | Olive Oil Substrate Solution |
| C | 95% Ethanol stored at 4° C. |
| D | 0.9% (w/v) Thymolphthalein Indicator |
| | Solution |
| E | 50 mM Sodium Hydroxide Solution |
| F | Lipase Enzyme Solution in Water |

Part 1: Preparation of Assay of Enzyme Activity

Test Solution Preparation: After 2.50 mL of water, 1.00 mL of Reagent A, and 3.00 mL of Reagent B were added to a 50 mL Erlenmeyer flask labeled “TEST,” the mixture was equilibrated to 37° C. After 1.00 mL of Reagent F was added thereto, the mixture was mixed and incubated at 37° C. for 30 minutes. After 20 mL of Reagent C and 4 drops of Reagent D were added thereto, the mixture was titrated with Reagent E to a light blue endpoint.

Blank Solution Preparation: Into a separate 50 mL Erlenmeyer flask labeled “BLANK” was added 2.50 mL of water, 1.00 mL of Reagent A, and 3.00 mL of Reagent B.

After incubating the blank mixture at 37° C. for 30 minutes, 20 mL of Reagent C, 1.00 mL of Reagent F, and 4 drops of Reagent D were added thereto. The mixture was then titrated with Reagent E to a light blue endpoint.

Part 2: Preparation of Test Solution: An appropriate amount of Formulation 1 as set forth in Table A above was weighed into a 50 mL Erlenmeyer flask labeled “Sample” such that the amount of the formulation would be in a 1:1 enzyme to clay ratio. After adding 2.50 mL of water, 1.00 mL of Reagent A, and 3.00 mL of Reagent B thereto, the resulting mixture was equilibrated to 37° C. After adding 1.00 mL of Reagent F to the flask, the mixture was thoroughly mixed and incubated at 37° C. for 60 minutes. After 20 mL of Reagent C and 4 drops of Reagent D were added thereto, the mixture was then titrated with Reagent E to a light blue endpoint.

This procedure was repeated, but with the substitution of Formulations 2 through 40, respectively, for that of Formulation 1.

Blank Solution Preparation: The Blank Solution was prepared as set forth above.

Part 3: Preparation of Synthetic urine

0.20 g KCl, 0.20 g NaSO₄, 0.08 g (NH₄)H₂PO₄, 0.01 g (NH₄)₂HPO₄, 0.02 g CaCl₂, 0.05 g MgCl₂, and 99.44 g H₂O were combined and stirred in a flask. When used, the synthetic urine is added to the test solution described above in Part 2 in place of the water.

Part 4: Calculation of Enzyme Inhibition Activity:

Enzyme Activity is defined by the following equation:
(Reagent E)(Molarity of Reagent E)(1000)(conversion)(df)
Wherein:
Reagent E=mL of Reagent E consumed with blank correction, i.e., a correction was made for the amount of NaOH (reagent E) that was required to bring the blank solution to a light blue endpoint in the titration.
Conversion=time conversion factor (e.g. “2” for 30 min. and “_{—}1” for 60 min.)
df=dilution factor=1

The enzyme activity inhibition for each formulation at issue is calculated as the difference between the enzymatic activity of the test solution of Part 1 with the enzymatic activity of each sample solution as set forth in Table A above.

Part 5: Testing of Clay Formulations for Enzyme Inhibition

The results of the enzyme inhibition tests for each of the respective formulations set forth in Table A above are shown in Table C below.

TABLE C
Results of Enzyme Inhibition Tests

| | | | % Enzyme Inhibition |
|---|---|---|---|
| Sample | Synthetic Urine** | Time*(minutes) | Activity |
| 1 | no | 30 | 24 |
| 1 | yes | 60 | 43 |
| 2 | no | 30 | 0 |
| 3 | yes | 60 | 31 |
| 4 | yes | 60 | 30 |
| 5 | no | 30 | 15 |
| 5 | yes | 60 | 19 |
| 6 | yes | 60 | 28 |
| 7 | no | 60 | 46 |
| 8 | yes | 60 | 20 |
| 9 | yes | 60 | 39 |
| 10 | yes | 60 | 28 |
| 11 | yes | 60 | 37 |
| 12 | yes | 60 | 39 |
| 13 | yes | 60 | 28 |
| 14 | yes | 60 | 35 |
| 15 | yes | 60 | 24 |
| 16 | yes | 60 | 40 |
| 17 | yes | 60 | 46 |
| 18 | yes | 60 | 42 |
| 19 | yes | 60 | 23 |
| 20 | yes | 60 | 41 |
| 21 | yes | 60 | 49 |
| 22 | yes | 60 | 33 |
| 23 | yes | 60 | 26 |
| 24 | yes | 60 | 41 |
| 25 | yes | 60 | 32 |
| 26 | yes | 60 | 46 |
| 27 | yes | 60 | 22 |
| 28 | yes | 60 | 41 |
| 29 | yes | 60 | 39 |
| 30 | yes | 60 | 43 |
| 31 | yes | 60 | 44 |
| 32 | yes | 60 | 38 |
| 33 | yes | 60 | 30 |
| 34 | yes | 60 | 21 |
| 35 | yes | 60 | 21 |
| 36 | yes | 60 | 30 |
| 37 | yes | 60 | 15 |
| 38 | no | 60 | 0 |
| 39 | no | 60 | 33 |
| 40 | no | 60 | 4 |

*Time is measured from the preparation of the final solution as set forth in Part 2 until the time at which inhibition is measured.
**2.5 ml of synthetic urine was added at time = 0

As particularly shown by Samples 7 through 9, this Example demonstrates that the compositions of this invention are effective at inhibiting enzymes known to cause diaper rash. More specifically, Sample 7, which is a clay-containing formulation without a peptizing agent, effectively inhibits enzymatic activity in the absence of urine. However, the clay's ability to inhibit enzymes is significantly decreased when exposed to urine as shown in Sample 8. As shown in Sample 9, the addition of a peptizing agent to the clay-containing formulation counteracted the deleterious affect of urine. This efficacy to inhibit enzymatic activity even in the presence of urine was observed for gels/suspension compositions (Sample 9), solid compositions (Sample 11), and emulsion compositions (Samples 31, 33, 34, 35). Therefore, this example showed that the compositions of the invention were effective at reducing the activity of the enzymes, even in the presence of urine.

As shown in Samples 39 and 40, emulsifiers containing several moles of ethylene oxide, i.e. greater than about 20 moles, appeared to be effective in enzyme inhibition, but were comparatively less effective than non-ethoxylated emulsifiers. See Sample 21.

The preferred composition of WO 97/38735, which was tested in Sample 37 above, possessed a relatively inferior enzyme inhibition activity relative to that of the compositions of the present invention, e.g., Samples 21 and 31. In addition, Samples 33 through 36 showed that the inclusion of short chain hydrocarbons, e.g. cosmetic esters commonly used in aesthetically pleasing personal care emulsions such as capric/caprylic triglyceride, isopropyl myristate, and isoparaffin, in the composition of the present invention resulted in efficacious enzyme inhibition, which is contrary to the teachings of WO97/38735. Further, the prior art emulsifier system of WO 97/38735, as tested above in Sample 38, was not efficacious in an oil in water type formulation with a water swellable clay. By contrast, as shown in Sample 32, the water in oil emulsifier system of the present invention is comparatively more efficacious in enzyme inhibition.

1. A topical composition comprising a swellable clay or adduct thereof, and a peptizing agent.

2. The composition of claim 1 wherein said composition is a solid composition.

3. The composition of claim 1 wherein said composition is a cosmetic suspension composition.

4. The composition of claim 1 wherein said composition is an oil-in-water composition further comprising an oil and an aqueous carrier.

5. A method for reducing the enzymatic activity of one or more enzymes selected from the group consisting of lipases, esterases, ureases, proteases, and combinations of two or more thereof present on the external skin, comprising topically applying to mammalian skin a composition of claim 1.

6. The method of claim 5 wherein said one or more enzymes comprises lipase.

7. The method of claim 5 wherein said one or more enzymes comprises a protease.

8. The method of claim 7 wherein said protease is selected from the group consisting of trypsin, chymotrypsin, elastase, carboxypeptidase, aminopeptidase, and combinations of two or more thereof.

9. The method of claim 8 wherein said protease comprises trypsin.

10. The method of claim 8 wherein said protease comprises chymotrypsin.

11. The method of claim 8 wherein said protease comprises elastase.

12. The method of claim 8 wherein said protease comprises carboxypeptidase.

13. The method of claim 8 wherein said protease comprises aminopeptidase.

14. The method of claim 5 wherein said one or more enzymes comprises an esterase.

13. The method of claim 5 wherein said one or more enzymes comprises an urease.

14. An article comprising a substrate containing a composition of claim 1.
